(19) United States
(12) Patent Application Publication
(10) Pub. No.: US 2017/0260255 A1
(43) Pub. Date: Sep. 14, 2017

(54) FUSION PROTEIN AND PURIFYING METHOD

(71) Applicant: Jörg LABAHN, Tostedt (DE)

(72) Inventors: Jörg LABAHN, Tostedt (DE); Johann KUBICEK, Köln (DE); Frank SCHÄFER, Düsseldorf (DE)

(21) Appl. No.: 15/509,105

(22) PCT Filed: Sep. 7, 2015
(86) PCT No.: PCT/EP2015/070393

§ 371 Date: Mar. 6, 2017

(30) Foreign Application Priority Data
Sep. 5, 2014 (DE) 10 2014 217 793.6

(51) Int. Cl. C07K 16/00; C07K 1/22; C12N 9/36; C07K 14/435

(52) U.S. Cl. CPC C07K 16/00; C07K 14/43595; C07K 1/22; C12N 9/2462; C12Y 302/01017; C07K 2319/20; C07K 2319/60; C07K 2317/565

(57) ABSTRACT

The invention relates to, inter alia, a fusion protein comprising a protein of interest and an affinity tag which binds lysozyme. Lysozyme can be used to purify, precipitate, or support the crystallization of such a fusion protein. The invention further relates to a binding agent which specifically binds a target compound, wherein the binding agent has a CDR3 region which is derived from a single-domain antibody but which does not comprise framework regions or other elements for stabilizing the CDR3 region, and wherein the binding agent binds the target compound via the CDR3 region.

### FIELD OF THE INVENTION

The present invention pertains to novel binding agents suitable for binding a target compound. Said binding agents are in particular useful in therapy and as detection agents for detecting a target compound, e.g. in assays or for diagnostic purposes. Additionally, these binding agents can be used as affinity tag.

Furthermore, the present invention pertains to a capture system comprising an affinity tag and an affinity ligand useful for purifying a protein of interest. Furthermore, specific uses of the respective system are described, in particular for the crystallization or purification of a protein of interest.

### BACKGROUND OF THE INVENTION

There is a continuous need in the prior art to provide novel binding agents having a binding affinity to a target compound. In particular, there is a need for small binding compounds which can be easily produced synthetically and/or recombinantly, and which despite their small size can specifically bind a target compound. Respective small binding compounds would also provide the advantage that they might be able to pass barriers in the body such as the blood-brain barrier which cannot be passed by conventional binding agents due to their size (e.g. antibodies). Respective small binding agents would have considerable advantages in the therapeutic and diagnostic field.

Therefore, it is an object of the present invention to provide compounds which specifically bind a target compound but which are smaller in size than e.g. antibodies or commonly used antibody fragments.

The recombinant DNA technology has enabled the production of desired proteins of interest in host cells. Such host-produced polypeptides typically are separated from host cell proteins prior to their use in order to obtain them in pure form. Affinity chromatography based methods are often preferred for protein purification. Affinity chromatography can be used to purify proteins from complex mixtures with high yield. Affinity chromatography is based on the ability of proteins to bind non-covalently but nevertheless specifically for example to a ligand for the desired protein that is immobilised to a support, for example to an antibody that binds the protein to be purified. When the protein of interest has affinity to a metal ion, isolation can be performed using metal affinity chromatography, as is for example the case in IMAC methods.

Furthermore, a protein of interest is often purified by expressing the protein of interest as fusion protein, wherein the fusion protein comprises the protein of interest and one or more affinity tags. The affinity tag allows the protein of interest to be purified using generalized protocols in contrast to highly customized methods associated with conventional chromatography. These tags provide the superior advantage of e.g. traditional metal affinity tags (for example when using a poly-His-tag), namely suitability for using large scale purification at low cost, and possible purification under denaturing concentrations etc. Several purification tags are known in the prior art, such as for example the poly-His-tag, a streptavidin tag, a SBP-tag, a GST-tag or similar purification tags which enable the isolation of various proteins via the same affinity tag.

Despite the numerous available affinity tags, there is still a need in the prior art for improved purification methods and in particular methods which allow to purify the protein of interest by using an affinity ligand, which can be either bound to a solid support or which allows to purify the protein of interest without the use of a solid support.

Therefore, it is an object of the present invention to provide a method for purifying a protein of interest.

Furthermore, it is an object of the present invention to provide means for supporting the crystallization of a protein of interest.

### SUMMARY OF THE INVENTION

One aspect of the present invention is based on the surprising finding that the isolated CDR3 region of heavy chain antibodies, in particular the CDR3 region derived from camelid antibodies (also referred to as CDR3 region), can be used as binding agent for binding a target compound even if the stabilising framework regions of the heavy chain antibody (and/or other commonly used stabilising structures) are removed, respectively are not used. This finding is very surprising because so far it was assumed that the stabilising framework regions or similar stabilising means are mandatory to provide the CDR3 region with the right conformation, respectively 3D structure in order to achieve binding of the CDR3 region to its target compound. However, it was now found that the CDR3 region alone—without conformation stabilising structures—is able to bind a target compound basically with the same specificity and/or affinity as the heavy chain antibody the CDR3 region is derived from. This finding allows to provide binding agents, which are even smaller in size than common heavy chain antibodies but which still bind their target compound with the desired specificity and/or affinitiy.

According to one aspect, the present invention therefore provides a binding agent specifically binding a target compound, wherein said binding agent comprises a CDR3 region derived from a heavy chain antibody but does not comprise framework regions for stabilising the CDR3 region or other stabilising structures for stabilising the CDR3 region and wherein said binding agent binds the target compound via the CDR3 region. Also provided is a pharmaceutical composition comprising a respective binding agent.

According to a further major aspect, the present invention is based on the concept to use a specific affinity tag based system which allows to purify, precipitate and/or crystallize a protein of interest. Said system is based on the use of lysozyme as affinity ligand and the use of an affinity tag which binds lysozyme. The inventors found that this specific combination of an affinity tag that binds lysozyme and lysozyme as affinity ligand provides considerable advantages when purifying, precipitating and/or crystallizing a protein of interest.

Furthermore, a fusion protein is provided which comprises a protein of interest and an affinity tag which specifically binds lysozyme. Preferably, said fusion protein is recombinantly produced.

Additionally, a method is provided for purifying a fusion protein comprising a protein of interest and an affinity tag which specifically binds lysozyme, said method comprising a step of using lysozyme as affinity ligand which binds to the affinity tag of the fusion protein. Thereby, a complex is formed which comprises lysozyme and the fusion protein to be purified from the sample. Said complex can then be separated from the remaining sample and the fusion protein can be released from the complex. Lysozyme can be added in free form to the sample which comprises the fusion protein, or it can be present immobilised to a support, such as e.g. a column.

Thus, the present invention also pertains to a complex comprising lysozyme as affinity ligand, wherein the lysozyme is bound to a fusion protein, which comprises a protein of interest and an affinity tag which binds lysozyme.

Furthermore, the present invention pertains to an expression vector for expressing a fusion protein, wherein said fusion protein comprises a protein of interest and an affinity tag which specifically binds lysozyme. Furthermore, a host cell is provided comprising a respective expression vector.

Furthermore, the present invention pertains to the use of lysozyme for supporting and/or enabling the crystallization of a fusion protein which comprises a protein of interest and an affinity tag which binds lysozyme.

Furthermore, the present invention pertains to the use of lysozyme for purifying a fusion protein which comprises a protein of interest and an affinity tag which binds to lysozyme.

Furthermore, the present invention pertains to the use of lysozyme for precipitating a fusion protein, which comprises a protein of interest and an affinity tag which binds lysozyme.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

The inventors have found that in contrast to the beliefs in the prior art, it is possible to use the CDR3 region derived from a heavy chain antibody without its framework regions or other conformation stabilising structures as binding agent for binding a target compound.

Therefore, according to one aspect of the present invention, a binding agent specifically binding a target compound is provided, wherein said binding agent comprises a CDR3 region derived from a heavy chain antibody but does not comprise the corresponding framework regions or other structures stabilising the CDR3 region and wherein said binding agent binds the target compound via the CDR3 region.

The CDR3 region according to the invention is derived from a heavy chain antibody. The CDR3 region is preferably derived from a heavy chain antibody of camelids (such as dromedaries, camels and alpacas) or may also be derived from a heavy chain antibody from other animals which produce respective antibodies such as e.g. sharks. A CDR3 region is “derived” from such a heavy chain antibody if the CDR3 region possesses a homology or identity over its entire length with the corresponding CDR3 region of the reference heavy chain antibody of at least 80%, at least 85%, at least 90%, at least 93%, at least 95%, at least 97%, at least 98%, at least 99% or 100%. The CDR3 region that is specific for the target compound can be e.g. obtained from the above mentioned antibodies. Methods for obtaining respective heavy chain antibodies against a target compound are known to the skilled person e.g. by using immunization methods that are known in the art. E.g. the dromedar, camel or shark can be immunized with the desired target compound as antigen and the mRNA coding for heavy chain antibodies can be subsequently isolated. By reverse transcription and polymerase chain reaction, a gene library of single domain antibodies containing several million clones can be produced. Screening techniques such as phage display and ribosome display may also be used to identify the clones binding the antigen. It is also within the scope of the present invention to obtain suitable binding CDR3 regions from heavy chain antibody libraries by utilizing screening methods. Once a suitable CDR3 region was identified, it can also be produced synthetically due to its short size. This is a considerable advantage over prior art binding agents which, due to their size, must be usually produced recombinantly.

According to one embodiment, said CDR3 region has a length of 10 to 30, preferably 12 to 25 amino acids. The CDR3 region of respective heavy chain antibodies possesses the extraordinary capacity to form fingerlike extensions that can extend into cavities on antigens, e.g. into the active site crevice of an enzyme. The CDR3 region can form e.g. convex extensions or protrusions which can e.g. occupy the cleft of a target compound. Thus, according to one embodiment, the CDR3 region binds to a cleft, pocket or canyon of a target compound. The unstabilized CDR3 region according to the present invention penetrates into the cleft, pocket or canyon of the target compound thereby becoming stabilized by the molecular interactions between the CDR3 region and the lining amino acids of the cleft, pocket or canyon. According to one embodiment, said CDR3 region can mimic the binding of the natural substrate to the pocket, cleft or canyon of the target compound, to which the CDR3 region binds to.

According to one embodiment, said CDR3 region is not cyclised by intramolecular bonds such as disulfide bonds. According to one embodiment, the CDR3 region is devoid of amino- and/or carboxy-terminal amino acid residues which would allow cyclization and thus the stabilisation of the CDR3 region. According to one embodiment, the CDR3 region is devoid of cysteine residues. Thus, in this embodiment the CDR3 region does not comprise a cysteine residue. A cysteine residue in the CDR3 region increases the risk of unspecific interactions via the cysteine residue (e.g. by forming disulfide bonds). Therefore, it is preferred to remove or replace any cysteine residues that are present in the CDR3 region that is used for binding. E.g. a cysteine residue present in the CDR3 region can be replaced by other amino acids, such as e.g. serine or alanine or other suitable amino acids. It is preferred to choose an amino acid for substitution which preserves or even improves the binding capability.

According to one embodiment, the binding agent consists of said CDR3 region. However, it is within the scope of the present invention to conjugate respectively to attach said CDR3 region to other compounds and/or structures, e.g. to compounds which extend the half-life of the CDR3 region such as e.g. HSA, HES or PEG, marker compounds or cytotoxic agents. Moreover, the attachment to other protein or peptide compounds is possible wherein e.g. the CDR3 region is conjugated or fused via a synthetic linker or via a glycine or alanine linker or by other connecting means to a fusion construct. The conjugation and/or attachment can be covalent or non-covalent. However, according to one embodiment, said conjugation does not result in a stabilisation of the conformation of the CDR3 region that is used as binding agent.

According to a preferred embodiment, said binding agent binds its target compound via the CDR3 region with the same specificity and/or affinity as the heavy chain antibody it is derived from which comprises the corresponding framework regions. As discussed before, it was surprisingly found by the inventors that the CDR3 region of heavy chain antibodies which is used according to the present invention for binding the target compound maintains its binding specificity, despite the fact that the conformation of the CDR3 region is not stabilised by the framework regions or other stabilising structures. This has the advantage that the binding agent can be designed smaller and furthermore, its production is considerably simplified, because it may be produced synthetically and it is not necessary e.g. to cyclise the CDR3 region or add conformation stabilising structures thereto.

According to one embodiment, said binding agent binds via said CDR3 region lysozyme as target compound. As discussed, the binding agent may consist of the CDR3 region.

For binding lysozyme said binding agent comprises according to one embodiment a CDR3 region which comprises or consists of a sequence which is selected from the group consisting of

SEQ ID NO 1:
DSTIYASYYECGHGLSTGGYGYDS
SEQ ID NO 2:
DTSTWYRGYCGTNPNYFSY
SEQ ID NO 3:
GWSSLGSCGTNRNRYNY
SEQ ID NO 4:
GYRNYGQCATRY
SEQ ID NO 5:
GYRNYGQSATRY
SEQ ID NO 6:
TRKYVPVRFALDQSSYDY

The examples of the present application demonstrate that the above isolated CDR3 regions of heavy chain antibodies are capable of binding lysozyme and thus can be used as binding agents for lysozyme. These binding agents which as discussed may also consist of one of the above sequences, can be advantageously used also as affinity tag, having an affinity to lysozyme. This is described subsequently in further detail.

Also provided with the present invention is a pharmaceutical or diagnostic composition, comprising a binding agent according to the present invention. Due to the small size of the binding agent, a respective pharmaceutical or diagnostic composition has several advantages. First of all, their simple and safe production is advantageous for therapeutic and diagnostic uses. Furthermore, the small size of the binding agent allows to e.g. penetrate barriers in the body such as e.g. the blood-brain barrier that can not be penetrated by larger molecules such as antibodies. The binding agent according to the present invention can be used analogous to conventional antibodies and binding agents in therapy and diagnosis. E.g. the binding agent can be used to detect a target compound, to thereby allow a diagnosis based on the presence or absence of the target compound.

Furthermore, the binding agent according to the present invention can be advantageously used as affinity tag, which allows the easy isolation of a protein of interest if said affinity tag is fused to the protein of interest. Such recombinant fusion can be achieved e.g. by expressing the protein of interest and the affinity tag as fusion construct. Thus, the present invention also provides according to one embodiment a fusion protein comprising a protein of interest and an affinity tag, wherein the affinity tag comprises or consists of a CDR3 region derived from a heavy chain antibody but which does not include framework regions for stabilising the CDR3 region. Preferably, said CDR3 region also does not comprise any other stabilising structures. As discussed above, the CDR3 regions derived from respective heavy chain antibodies have several advantages that make them particularly suitable for use as affinity tag. They are small and surprisingly do not require the stabilising framework regions or other conformation stabilising structures in order to bind a target compound and thus an affinity ligand with high specificity. Thus, they can be easily expressed as fusion protein together with the protein of interest. As discussed above, said CDR3 region which exerts the binding function and thus can serve as affinity tag preferably has a length of 10 to 30, preferably 12 to 25 amino acids. According to one embodiment, said CDR3 region forms a fingerlike extension or protrusion that can extend into the cavity, cleft or pocket of an affinity ligand, e.g. the active site crevice of an enzyme. Thus, according to one embodiment, said CDR3 region binds to a cleft, pocket or canyon of a target compound which is used as affinity ligand. Binding is achieved as the CDR3 region e.g. penetrates into the cleft, pocket or canyon of the target compound thereby becoming stabilized by the molecular interactions between the CDR3 region and the lining amino acids of the cleft, pocket or canyon. The affinity ligand thus preferably comprises a respective cleft, pocket or canyon and most preferably is an enzyme such as lysozyme. This preferred embodiment will also be described in further detail below.

The affinity tag is preferably unrelated to the protein of interest and therefore, is naturally not expressed as a corresponding fusion protein.

The recombinant fusion protein can then by isolated via the CDR3 region which serves as affinity tag and which specifically binds to the respective affinity ligand. Said affinity ligand can e.g. be immobilised to a solid support such as e.g. a column or it can also be directly added in free form to a composition comprising the fusion protein in order to precipitate the fusion protein as it is described subsequently for the preferred example lysozyme as affinity ligand.

According to a further aspect, the present invention pertains to a novel affinity tag/affinity ligand system which is based on the use of lysozyme as affinity ligand and an affinity tag which specifically binds to lysozyme. This novel affinity tag/affinity ligand system, which is based on the use of lysozyme as affinity ligand, has several advantages and broad applications in the field of protein purification, protein precipitation and/or protein crystallization.

According to a further aspect, a fusion protein is provided, which comprises a protein of interest and an affinity tag that is capable of binding lysozyme. A respective fusion protein, which can be e.g. recombinantly produced, can be easily purified with the method according to the present invention by using lysozyme as affinity ligand. The lysozyme binding affinity tag is not naturally associated with the protein of interest but instead the fusion construct is e.g. generated by recombinant DNA-technology.

The fusion protein according to this aspect of the present invention comprises a specific affinity tag which binds to lysozyme, thereby allowing a specific affinity-based isolation of the fusion protein. According to one embodiment the affinity tag which binds lysozyme is located either at the N-terminus or at the C-terminus of the fusion protein. Preferably, the affinity tag according to the present invention is located at the C-terminus. The location of the affinity tag, however, may depend on the fusion protein to be expressed and its intended use. One of the advantages of using an N-terminal located affinity tag is that the yield of the fusion protein expressed is increased as a reliable context for efficient translation is provided. However, the use of an N-terminal affinity tag does not always lead to the purification of full length proteins as aberrant translation products, which do not comprise the full length protein of interest are due to the N-terminal affinity tag also co-purified. Thus, for most applications an affinity tag that is located at the C-terminus will be advantageous, because the purification of the full length fusion protein is increased compared to N-terminal affinity-tag fusions.

According to a preferred embodiment, the affinity tag binds to the active site of lysozyme. The inventors found, that it is advantageous if the affinity tag binds to the active site of lysozyme, as thereby a high specificity to the affinity ligand lysozyme is achieved. Furthermore, binding of the affinity tag to the active site of lysozyme allows to use mild elution conditions during the purification of the fusion protein, e.g. by using sugars that are bound by lysozyme. Respective sugars can displace the fusion protein respectively the affinity tag from the active site of lysozyme thereby releasing it from the complex. Thereby, the fusion protein can be separated from the affinity ligand lysozyme as it is also described subsequently in conjunction with the purification method according to the present invention. This supports the elution.

According to one embodiment, the affinity tag comprises or consists of an immunoglobulin molecule or a functional fragment thereof, which binds lysozyme. In particular, the immunoglobulin molecule can be an antibody.

The term “antibody” particularly refers to a protein comprising at least two heavy chains and two light chains connected by disulfide bonds. The term “antibody” includes naturally occurring antibodies as well as all recombinant forms of antibodies, e.g., antibodies expressed in prokaryotes, unglycosylated antibodies, humanized antibodies, and chimeric antibodies. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The constant regions of the antibodies may mediate the binding of the immunoglobulin to host tissue or factors, including to various cells of the immune system (e.g., effector cells) and the first component (C1q) of the classical complement system.

Furthermore, the term immunoglobulin molecule or functional fragment thereof in particular includes but is not limited to a protein or glycoprotein which is derived from an antibody and is capable of binding to the same antigen, in particular to the same epitope as the antibody. Thus, a fragment or derivative of an antibody as used herein generally refers to a functional fragment or derivative that can bind to the antigen. In a particularly preferred embodiment, the fragment or derivative of an antibody comprises a heavy chain variable region. It has been shown that the antigen-binding function of an antibody can also be provided by fragments of a full-length antibody or derivatives thereof. Examples of fragments or derivatives of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. These antibody fragments and derivatives can be obtained using conventional techniques known to those with skill in the art.

Preferably, the affinity tag is derived from a heavy-chain antibody. Heavy-chain antibodies are well-known in the prior art and can be for example obtained from camelids as is described in detail above. The use of respective heavy-chain antibodies has the advantage that they preferably bind to the active site of the enzyme, against which they were raised. Therefore, respective heavy-chain antibodies are particularly suitable as affinity tag for binding the active site of lysozyme. Furthermore, the use of heavy-chain antibodies or functional i.e. lysozyme binding fragments thereof has the advantage that these affinity tags are comparably small what is advantageous for an affinity tag because the producing host cell only needs to invest little capacities in the production of the affinity tag, thereby ensuring the expression of the protein of interest with high yield.

According to a preferred embodiment, the affinity tag comprised in the fusion protein has one or more of the characteristics of the above described binding agent that comprises a CDR3 region of a heavy chain antibody. Thus, preferably, the affinity tag is a CDR3 region derived from a heavy chain antibody. The characteristics and preferred embodiments of said CDR3 region are described in detail above and it is referred to the above disclosure which also applies here. As discussed above, the isolated CDR3 region of respective heavy chain antibodies has several advantages that makes it suitable for use as an affinity tag. It is small and surprisingly does not need to be stabilized by framework regions or other structures in order to be able to bind a target compound and thus an affinity ligand with high specificity. Thus, such an CDR3 region can be easily expressed as fusion protein together with the protein of interest and can serve here as affinity tag. As discussed above, said CDR3 region which exerts the binding function to the affinity ligand preferably has a length of 10 to 30, preferably 12 to 25 amino acids. According to one embodiment, said CDR3 region forms at least one fingerlike extension or protrusion that can extend into the cavity, cleft or pocket of the affinity ligand lysozyme. Thus, according to one embodiment, said CDR3 region binds to a cleft, pocket or canyon of a lysozyme which is used as affinity ligand. Binding is achieved as the CDR3 region e.g. penetrates into the cleft, pocket or canyon of lysozyme thereby becoming stabilized by the molecular interactions between the CDR3 region and the lining amino acids of the cleft, pocket or canyon.

According to one embodiment, the affinity tag comprises a sequence that is selected from the group consisting of

SEQ ID NO 1:
DSTIYASYYECGHGLSTGGYGYDS
SEQ ID NO 2:
DTSTWYRGYCGTNPNYFSY
SEQ ID NO 3:
GWSSLGSCGTNRNRYNY
SEQ ID NO 4:
GYRNYGQCATRY
SEQ ID NO 5:
GYRNYGQSATRY
SEQ ID NO 6:
TRKYVPVRFALDQSSYDY

or the affinity tag consists of a corresponding sequence. As is shown by the examples, the respective isolated, unstabilised CDR3 regions bind lysozyme specifically and thus are suitable for use as affinity tag. Compared to SEQ ID NO: 4, a cysteine was replaced by serine in SEQ ID NO: 5.

According to one embodiment, the fusion protein according to the present invention comprises a proteolytic cleavage site. Preferably, said proteolytic cleavage site separates the protein of interest from the affinity tag and thus allows to remove the affinity tag from the fusion protein in order to obtain the protein of interest without the affinity tag.

Preferably, the proteolytic cleavage site is a peptide sequence which typically comprises about 5-30 amino acids and represents a recognition motif including a cleavage site. Preferably, a proteolytic cleavage site is used that is not recognized by a protease that is expressed by the host cell in which the fusion protein is expressed using an expression vector. This is to ensure that the fusion protein is expressed with the affinity tag thereby allowing the isolation of the fusion protein and not being cleaved by the proteases of the host cell in advance. There are a variety of proteases that can be used later for liberating respectively splitting off the affinity tag from the fusion protein, so that subsequently the protein of interest can be purified without the affinity tag.

Furthermore, the present invention pertains to an expression vector for expressing a fusion protein which comprises a protein of interest and an affinity tag which binds lysozyme. As described above, according to one embodiment, the affinity tag may comprise or consist of a CDR3 region that is derived from a heavy chain antibody but which is not associated with the corresponding framework regions for stabilising the CDR3 region. Said affinity tag can bind lysozyme. Said expression vector enables the expression of a fusion protein which comprises a protein of interest and a lysozyme binding affinity tag according to the present invention.

The expression vector comprises one or more functional elements, which enable the expression of the fusion protein in a host cell. Said functional elements may be selected from the group consisting of a promoter, an enhancer, a transcription termination signal sequence, a poly(A)site, an intron for enhancing the expression of the fusion protein, and a secretory signal sequence for secreting the fusion protein. A secretory signal sequence is a peptide sequence which typically comprises about 10-30 amino acids, preferably about 15-30 amino acids. The secretory signal sequence is usually located at the N-terminus of the expression fusion protein and enables the secretion of the fusion protein, i.e. the passage through a cell membrane, for example into the cell culture or extracellular medium or into the periplasmatic space. In this course the signal sequence is usually cleaved off, so that the fusion protein is secreted. Several suitable secretory signal sequences are known in the art which allow the secretion from either eukaryotic or prokaryotic cells. Suitable secretory signal sequences are for example described in WO 2008/000445, the disclosure of which is hereby incorporated by reference.

Suitable promoters which allow the expression of the fusion proteins in prokaryotic and/or eukaryotic cells are also well-known to the skilled person and therefore do not need further description. Suitable promoters include, e.g. the CMV promoter, the SV40 promoter, the lacZ-promoter, the ubiquitin promoter, regulatory promoters or constitutive promoters and the like.

Enhancer sequences for upregulating expression of a fusion protein are also known to the skilled person. Enhancer sequences may be obtained from any eukaryotic or prokaryotic host, preferably in association with the corresponding promoter that is used. Enhancer elements may include CMV-enhancers, one or more glucose dependent elements and the like. Respective enhancer elements are very well-known in the art as well as promoter/enhancer combinations which may also be used according to the teachings of the present invention.

Furthermore, the expression vector as described above may contain further transcription and/or translation signals, preferably recognized by the appropriate host, such as e.g. transcriptional regulatory and translational initiation signals. Transcription and/or translation signals may be obtained from any eukaryotic, prokaryotic, viral, bacterial, fungal or plant origin, preferably from human or animal hosts, for example mammalian hosts, preferably in association with the corresponding promoters that are used. A wide variety of transcriptional and translational regulatory sequences may be employed for this purpose, depending upon the nature of the host cells. To the extent that the host cell recognizes the transcriptional regulatory and translational initiation signals contained in the expression vector according to the present invention, also the naturally occurring 5′ regions that are located adjacent to components of the fusion protein may be retained in the expression vector and can be employed for the translation regulation according to the present invention. However, also other regulatory signals may be used. The design of according expression vectors is known to the skilled person.

In addition to the regulatory sequences described above, the expression vector according to the present invention may comprise an origin of replication. Suitable origins of replication include for example ColE1, pSC101, SV40, ori pMP1 and M13 origins of replication. Depending on the host cell used, said origin of replication should be active in either prokaryotic or eukaryotic host cells or both.

Suitable host cells include e.g. prokaryotic or eukaryotic host cells, for example bacterial, fungal, plant, human and animal host cells. Preferred prokaryotic host cells may be for example derived from bacteria, such as Escherichia coli, B subtilis, salmonella, pneumococcus etc. or from algae, fungae etc. Furthermore, eukaryotic host cells can be used such as for example animal or human cells, for example rodent cells such as CHO cells. Cells from eukaryotic organisms are particularly preferred, if post-translational modifications, for example specific glycosylations of the protein of interest are required. The host cell may also be derived from yeast. Suitable host cells are also known to the skilled person.

Furthermore, the eukaryotic expression vector according to the present invention may comprise one or more selectable marker genes. Suitable selection markers are well-known in the prior art and may be e.g. selected from the group of eukaryotic or prokaryotic selection markers. Said selection markers may confer e.g. resistance against antibiotics such as e.g. ampicillin, hygromycin, kanamycin, neomycin and the like. Further selection markers include but are not limited to DHFR, GS and the like.

Also provided is a host cell comprising an expression vector according to the present invention. The expression vector was described in detail above and it is referred to the respective disclosure.

Also provided is a method for purifying a fusion protein which comprises a protein of interest and a binding agent according to the present invention as affinity tag, wherein an affinity ligand which binds the affinity tag of the fusion protein is used for purification of the fusion protein. The fusion protein and in particular the comprised affinity tag preferably have the characteristics as described above. It is referred to the above disclosure. As discussed above, the binding agent that is used as affinity tag comprises a CDR3 region that is derived from a heavy chain antibody but does not include framework regions and preferably also not other structures for stabilising the CDR3 region. According to one embodiment, the binding agent that is used as affinity tag consists of such a CDR3 region. The affinity ligand and the fusion protein form a complex, which can be separated from the remaining sample, thereby isolating the fusion protein. Suitable, to lysozyme binding sequences are disclosed above.

Also provided is a method for purifying a fusion protein which comprises a protein of interest and an affinity tag which binds lysozyme from a sample comprising said fusion protein. The fusion protein and in particular the affinity tag preferably have the characteristics as described above. It is referred to the above disclosure which also applies here.

One important characteristic of said method is that lysozyme is used as affinity ligand for binding the affinity tag of the fusion protein specifically. The affinity ligand lysozyme is bound by the affinity tag of the fusion protein, thereby forming a complex comprising the fusion protein and lysozyme. For purifying the fusion protein, said complex may be separated from the remaining sample.

As described above, the respective fusion protein can be easily purified by using lysozyme as affinity ligand. The lysozyme used can be a c-type, g-type or i-type lysozyme. Preferably the lysozyme is selected from the group of c-type lysozymes with the ability to cleave beta-(1,4)-glycosidic bonds between N-acetylmuramic acid and N-acetylglcosamine within peptidoglycans. More preferably, the c-type lysozyme is of mammalian, bird, reptile origin, more preferably of bird and most preferably of chick origin. The lysozyme used as affinity ligand can be obtained from natural sources or may also be produced recombinantly.

According to one embodiment, lysozyme is added to the sample comprising said fusion protein. The sample can be for example a lysate, preferably a cleared lysate, or a culture medium comprising the fusion protein. The addition of lysozyme to the sample has the effect that the fusion protein is precipitated in form of a complex which comprises the fusion protein and lysozyme. Accordingly, lysozyme is used in an amount, respectively concentration, resulting in precipitation of the complexes. This presumably leads to a conformation change that benefits the precipitation. This embodiment, wherein lysozyme is added in free form to the sample, has the advantage, that the fusion protein can be purified without the use of a solid matrix as it is usually used in regular affinity chromatographies, which are based on the use of affinity ligands. Therefore, this embodiment of the purification method according to the present invention which does not use a solid support for purification allows the cost efficient purification of a protein of interest by making use of the fact that lysozyme can precipitate a fusion protein, provided that the fusion protein comprises an affinity tag, in particular a CDR3 region of a heavy chain antibody, which binds to lysozyme.

However, according to one embodiment, the lysozyme that is used as affinity ligand is immobilized to a solid support. Also here a complex is formed at the support. A respective purification method can be performed according to the well-known principle of affinity systems (such as e.g. His-Tag based systems), in which the target is bound using affinity ligands immobilized to columns. Lysozyme can be bound directly or e.g. via a linker molecule to the solid support. Suitable solid supports which can be used in a respective affinity chromatography purification process are known in the prior art and thus, need no further description.

The remaining sample can then be separated from the formed complex. If desired, the complex can be washed prior to separating the fusion protein from the affinity ligand lysozyme.

According to a preferred embodiment, the fusion protein is separated from the lysozyme and thus is released from the complex. According to one embodiment, this release, herein also referred to as “elution”, is achieved by using an elution solution. Elution can be achieved by various ways. E.g. sugars, peptides, lysozyme binding affinity tags in free form or other agents may be used, that have an affinity against the binding site of lysozyme and therefore can displace the fusion protein that is bound via the affinity tag from the complex. According to one embodiment, which allows a very mild elution of the fusion protein, an elution solution is used, which comprises at least one sugar that is bound by the active site of lysozyme. This embodiment is particularly suitable, if an affinity tag is used, which binds to the active site of lysozyme. Such affinity tag is preferably a CDR3 region which is derived from a heavy chain antibody as described above. It is referred to the above disclosure which also applies here. The elution solution may contain the natural substrate of the enzyme, e.g. selected from the group of peptidoglycans, more specifically from the group of peptidoglycans comprising 1,4-beta-linkages between N-acetylmuramic acid and N-acetyl-D-glucosamine residues. Preferably, said sugar is comprised in excess in the elution solution to facilitate the release of the affinity tag from the active site of lysozyme. This is particularly preferred in case an affinity tag is used which binds to the active site of lysozyme, because said excess in sugar can easily displace the fusion protein from the lysozyme, whereby the fusion protein is released from the complex.

Also provided by the teachings of the present invention is a complex comprising lysozyme and a fusion protein, which comprises a protein of interest and an affinity tag which binds lysozyme. A respective complex can be for example beneficially used for crystallization of the comprised fusion protein, thereby allowing the analysis of the protein of interest. Lysozyme is a protein which can be easily crystallized and it was found, that it can be used for supporting the crystallization of a fusion protein comprising a protein of interest and an affinity tag which binds lysozyme. These characteristics allow even the crystallization of a protein of interest which is otherwise not or only difficult to crystallize. The complex according to the present invention can therefore also be used for that purpose. The use of a lysozyme binding CDR3 region of a heavy chain antibody as affinity tag as is described above is a preferred embodiment which has the advantage that the affinity tag is very small and thus, lowers the risk that the three dimensional structure of the protein of interest is altered.

A further aspect of the present invention pertains to the use of lysozyme for purifying a fusion protein which comprises a protein of interest and an affinity tag which binds lysozyme. The details of the respective purification method are described above and it is referred to the above disclosure.

The present invention furthermore pertains to the use of lysozyme for precipitating a fusion protein which comprises a protein of interest and an affinity tag which binds lysozyme. As is shown by the examples, binding of the fusion protein with the lysozyme binding affinity tag to lysozyme results in conformational changes, leading to a decreased solubility of said lysozyme-fusion-protein-complex, so that the complex then precipitates. The application is quick and comprehensive. Thus, the decreased solubility of the protein complex can be used for easily separating the complex from the remaining sample by various means such as for example centrifugation, sedimentation etc. As described above, a respective precipitation based purification is very efficient with respect to costs and time and allows the purification of the fusion protein without the need for affinity-based column chromatography.

Furthermore, a respective precipitation process is of advantage when detecting e.g. a protein in an assay. Here, a corresponding precipitation can for example be used to increase the local concentration of the protein to be detected, thereby increasing the sensitivity of the assay. It may also be used for quantifying a protein of interest by adding a second enzymatic or biochemical reaction to the precipitation process described above. Respective methods can also be used for diagnostic purposes.

Examples for the use of the present invention in diagnostic assays encompass e.g. the determination of the concentration of a protein or compound of interest in clinical samples, such as, e.g. enzymes indicative for a certain type of disease, inflammatory process or marker proteins from pathogens such as e.g. viruses, which are usually present only in low concentration. A precipitation process according to the invention would therefore increase the sensitivity of assays of this particular type.

The fusion protein preferably has one or more of the above described characteristics. It is referred to the respective disclosure.

The protein of interest can be of any kind. The term “protein” refers to a molecule comprising a polymer of amino acids linked together by peptide bonds. The term “protein” includes polypeptides of any length (e.g. having more than 50 amino acids) and peptides (e.g. 2-49 amino acids). The term includes polypeptides and/or peptides of any activity or bioactivity, including e.g. bioactive polypeptides such as enzymatic proteins or peptides (e.g. proteases, kinases, phosphatases), receptor proteins or peptides, transporter proteins or peptides, bactericidal and/or endotoxin-binding proteins, structural proteins or peptides, immune polypeptides, toxins, antibiotics, hormones, growth factors, vaccines and the like. Said polypeptide may be selected from the group consisting of peptide hormones, interleukins, tissue plasminogen activators, cytokines, immunoglobulins, in particular antibodies or antibody fragments or variants thereof. Said immunoglobulin can be of any isotype. Very often IgG molecules (e.g. IgG1) are produced or needed as therapeutic proteins. An antibody fragment is any fragment of an antibody comprising at least 20 amino acids from said whole antibody, preferably at least 100 amino acids and which has the capability to bind an antigen. The antibody fragment may comprise the binding region of an antibody such as e.g. a Fab fragment, a F(ab)2 fragment, multibodies comprising multiple binding domains such as e.g. diabodies, triabodies or tetrabodies, single domain antibodies or affibodies. An antibody variant is e.g. a derivative of an antibody or antibody fragment having the same binding function but e.g. an altered amino acid sequence. Said antibody and/or the antibody fragment may comprise a murine light chain, a human light chain, a humanized light chain, a human heavy chain and/or a murine heavy chain as well as active fragments or derivatives thereof. Hence, it can be e.g. murine, human, chimeric or humanized. The protein of interest is preferably not naturally associated with the affinity tag used in the fusion protein and preferably, is no heavy chain antibody or fragment thereof if a CDR3 region derived from a heavy chain antibody is used as affinity tag.

### EXAMPLES

### Example 1

The affinity of the lysozyme-binding peptides provided by the present invention, which can be used as affinity tag, was analysed. The amino acid sequence of the affinity tags P2-P6 corresponds to the sequences shown as SEQ ID NO 2-6 and are derived from a CDR3 region of a heavy chain antibody. The affinity tags P2 to P6 were fused N-terminally to GFP as model protein of interest, so that a fusion protein was created which comprises GFP (protein of interest) and an affinity tag binding to lysozyme (P2 to P6). Said GFP protein comprised an additionally a His Tag at the C-terminus. The affinity of the resulting GFP fusion proteins for lysozyme was determined. The results of the measurements are provided in the table below:

| | Dissociation | Calculated | Calculated | Standard |
|---|---|---|---|---|
| | kd (1/s) | ka/kd (1/M) | 1/KA KD (M) | deviation (KD) |
| P2-GFP | 1.26 × 10⁻³ | 8.41 × 10⁵ | 1.23 × 10⁻⁶ | 1.95 × 10⁻⁷ |
| P3-GFP | 8.26 × 10⁻⁴ | 1.92 × 10⁶ | 5.36 × 10⁻⁷ | 9.41 × 10⁻⁸ |
| P4-GFP | 5.77 × 10⁻⁴ | 3.36 × 10⁶ | 3.33 × 10⁻⁷ | 1.09 × 10⁻⁷ |
| P5-GFP | 2.68 × 10⁻³ | 6.16 × 10⁶ | 1.66 × 10⁻⁷ | 2.85 × 10⁻⁸ |
| P6-GFP | 2.91 × 10⁻³ | 1.08 × 10⁶ | 9.54 × 10⁻⁷ | 1.94 × 10⁻⁷ |

The results show, that the small affinity tags of the present invention which consist of the CDR3 region of a heavy chain antibody respectively are derived therefrom, can bind lysozyme with high affinity, despite the fact that they are not stabilised by framework regions or other stabilising structures. The affinity can be further increased by making appropriate amino acid substitutions in the peptide sequences of the affinity tag. This can be assisted, e.g. by molecular modelling.

### Example 2

Example 2 demonstrates that the lysozyme binding affinity tag according to the present invention can be used to precipitate and thus purify a fusion protein which comprises a protein of interest and said affinity tag. The fusion protein P5-GFP comprises at the N-terminus an affinity tag that is specific for lysozyme. In the affinity tag P5 (see SEQ ID NO: 5), a cysteine was replaced against a serine compared to the affinity tag P4 (see SEQ ID NO: 4). The fusion protein P5-GFP comprised additionally a C-terminal His tag and was expressed in E. coli.

Lysozyme was then used to precipitate the P5-GFP fusion protein. Here, two samples were tested. One sample comprised the cleared bacterial lysate (sample 1), while the other sample contained P5-GFP, which was pre-purified and thus concentrated using Ni-NTA superflow resin (Qiagen) following the instructions provided by the manufacturer (sample 2).

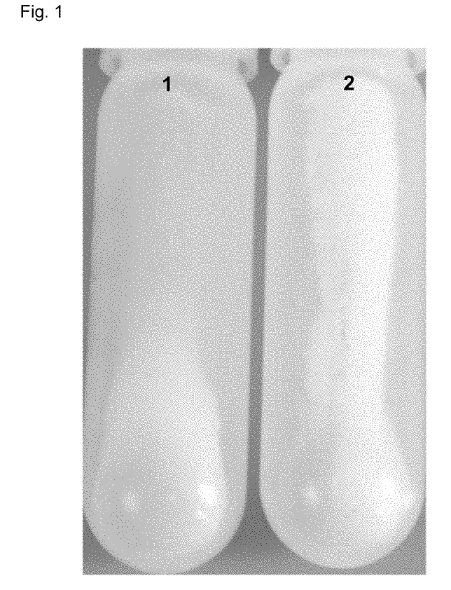

The mixture was then centrifuged. As is shown in FIG. 1, lysozyme precipitates the P5-GFP fusion protein. Binding of lysozyme to the affinity tag of the P5-GFP fusion protein results in a complex which precipitates due to a conformational change of lysozyme. The precipitated complex is visible as yellow pellet in FIG. 1. Sample 2 which comprised the via the additional His tag pre-purified P5-GFP fusion protein leads to a larger pellet due to the higher concentration of fusion protein. However, as it is shown by FIG. 1, the addition of lysozyme to the cleared lysate also results in a precipitation of the fusion protein and thus enables the quick and simple isolation of the fusion protein.

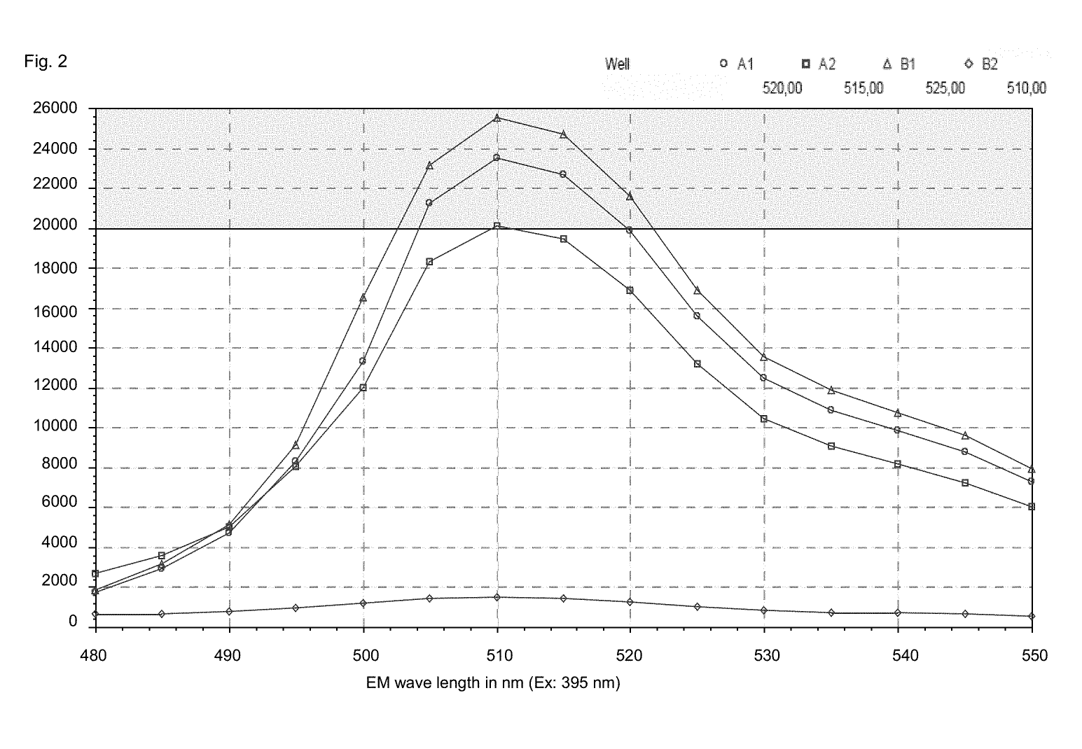

The obtained P5-GFP-lysozyme complexes were then purified by gel filtration and analyzed by SDS page and Coomassie staining as well as by photometric analysis of individual fractions at OD=509 nm. FIG. 2 shows the photometric analysis of individual fractions purified by gel filtration. The relative fluorescence was plotted as a function of the wavelength. Peak absorbance was observed at OD=509 nm, in accordance with the excitation-emission spectrum of GFP. Different concentrations were tested. The results show, that at a certain concentration, precipitation of the complexes occurs instantly. Thereby, more than 90% and even more that 95% of the protein of interest is precipitated.

### Example 3

The use and specificity of the affinity tag for lysozyme (Lysotag) for protein purification was assessed. 1000 μl of GFP-His, GFP-Strep and P5-GFP buffered in 100 mM NaCl, 50 mM Tris pH 7.5 were aliquoted into Eppendorf tubes and lysozyme was then added and the samples were incubated at room temperature. The addition of lysozyme resulted in an increased turbidity of the P5-GFP sample, which was not observed for the GFP-His and GFP-Strep samples. The samples were then centrifuged at 4° C. for 15 minutes, which resulted in pelletizing of the P5-GFP containing sample, but not for GFP-His and GFP-Strep.

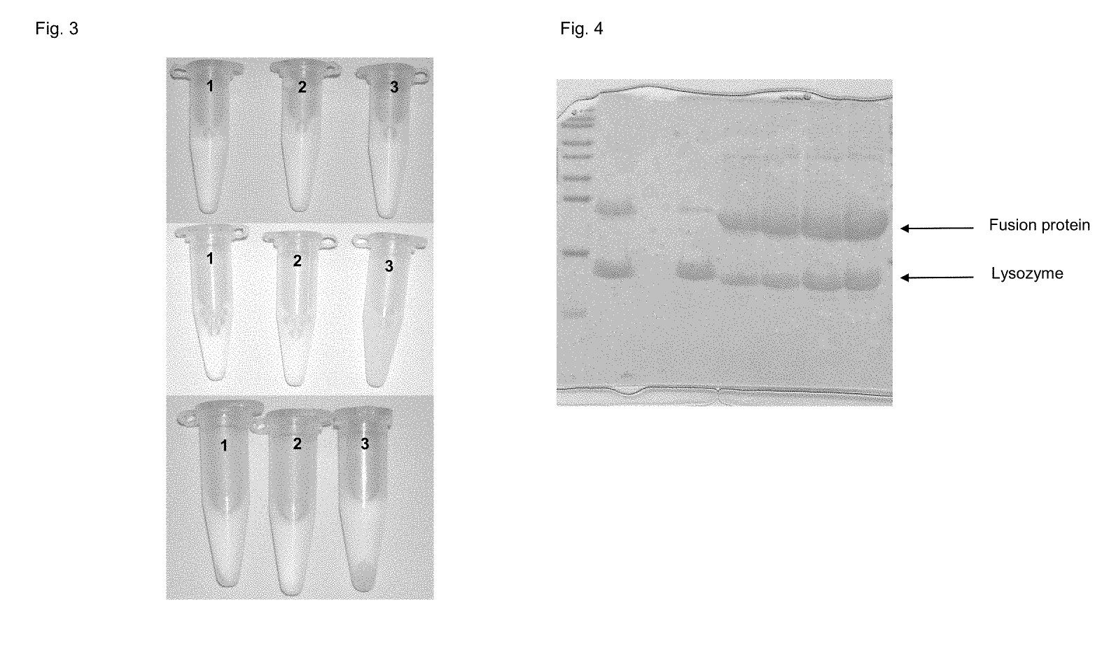

The results are shown in FIG. 3 which demonstrates the specificity of the precipitation of P5-GFP by the addition of lysozyme. (1) GFP-His, (2) GFP-Strep, (3) P5-GFP. A: samples prior to the addition of lysozyme. B: Samples after the addition of lysozyme. The P5-GFP sample appears turbid following the addition of lysozyme. C: Samples after centrifugation for at 4° C. at 12.000 g. The addition of lysozyme has resulted in the precipitation of the P5-GFP sample, which is evident from the yellow pellet after centrifugation. This pellet comprises the formed complexes. No effect was observed for the control samples. The His-GFP sample was clear also after centrifugation and for the Strep-GFP sample a white pellet showed after centrifugation. This demonstrates that the precipitation is specifically caused by the interaction of lysozyme with the P5-tag and is not induced by an unspecific protein aggregation, which is for example caused by the addition of lysozyme to the sample. It is noted that GFP does not become denatured during the precipitation process and retains its fluorescence throughout the whole purification process. GFP was moreover hardly detectable in the supernatant which underscores the efficiency of the method. Thereby a quick, cost-efficient and effective purification method is provided, which can be performed without support materials such as for example columns.

### Example 4

Further tests were performed in order to demonstrate that the precipitation is also not caused by an unspecific interaction between lysozyme and GFP, but is due to a specific interaction between the lysozyme and the affinity tag. Furthermore, it should be demonstrated that the concept of the present invention can be used with different proteins of interest.

In example 4, interferon alpha was used as protein of interest which was provided N-terminally with the lysozyme binding affinity tag P5 (see SEQ ID NO: 5). Thereby, a fusion protein was obtained, which comprises interferon-alpha (protein of interest) and a lysozyme binding affinity tag (P5). The addition of lysozyme led to an instant precipitation of the fusion protein, which comprises the lysozyme binding affinity tag. Precipitation of the complex was assisted by centrifugation so that a kind of pellet was produced, which comprises the formed complexes. The results of the gel electrophoresis are shown in FIG. 4.

In the first lane (adjacent to the marker), the sample comprising the lysozyme and the tagged fusion protein (before centrifugation) was applied onto the gel. As can be seen, said sample comprises lysozyme as well as the fusion protein, which comprises the affinity tag. In the third lane adjacent to the marker (the second lane is empty) the supernatant was applied. As can be seen, said supernatant comprises excess lysozyme and only trace amounts of the fusion protein. This demonstrates that almost all of the fusion protein comprising interferon alpha was precipitated efficiently by the addition of lysozyme. Adjacent thereto, different concentrations of the pellet were applied to the gel. As can be seen, the precipitated pellet comprises the interferon alpha containing fusion protein and lysozyme.

1. A fusion protein comprising a protein of interest and an affinity tag that binds lysozyme.

2. The fusion protein according to claim 1, wherein the affinity tag of the fusion protein has one or more of the characteristics of the CDR3 region.

3. A method for purifying a fusion protein according to claim 1 from a sample comprising said fusion protein wherein in said method lysozyme is used as affinity ligand and wherein the affinity tag of the fusion protein binds to lysozyme, thereby forming a complex.

4. The method according to claim 3, wherein
a) the lysozyme is contacted with the sample, to thereby precipitate the complex comprising the fusion protein and lysozyme or
b) the lysozyme is provided immobilized to a support and the fusion protein is bound via the lysozyme to the support.

5. The method according to claim 3, wherein the remaining sample is separated and optionally, the fusion protein is released from the complex.

6. The method according to claim 3, wherein the fusion protein is released by using an elution solution, which comprises a sugar that is bound by lysozyme.

7. A binding agent that specifically binds a target compound, wherein said binding agent comprises a CDR3 region that is derived from a heavy chain antibody but does not comprise framework regions or other elements for stabilising the CDR3 region and wherein said binding agent binds the target compound via the CDR3 region.

8. A binding agent according to claim 7, wherein the binding agent consists of said CDR3 region.

9. A binding agent according to claim 7, having one or more of the following characteristics:
a) the CDR3 region of the binding agent binds the target compound with the same specificity and/or affinity as a heavy chain antibody which comprises the framework regions;
b) the CDR3 region has a length of 10 to 30, optionally 12 to 25, amino acids;
c) the CDR3 region binds to a cleft, a pocket or a canyon of a target compound;
d) the CDR3 region is devoid of amino- and/or carboxy-terminal amino acid residues which would allow a cyclization;
e) the CDR3 region mimics the natural substrate binding to the pocket, cleft or canyon of the target compound, to which the CDR3 region binds, and/or
f) said CDR3 region does not comprise a cysteine residue.

10. A binding agent according to claim 7, wherein said binding agent binds via said CDR3 region lysozyme as target compound.

11. A binding agent according to claim 10, wherein said binding agent comprises a CDR3 region which comprises or consists of a sequence selected from the group consisting of SEQ ID NO: 1 to 6.

12. A fusion protein comprising a protein of interest and an affinity tag, wherein the affinity tag comprises a CDR3 region that is derived from a heavy chain antibody but does not include framework regions for stabilising the CDR3 region, wherein optionally the CDR3 region has one or more of the characteristics of the CDR3 region defined in claim 8.

13. Lysozyme to purify, precipitate or support the crystallization of a fusion protein according to claim 1.


SEQUENCE LISTING (6 sequences)

SEQ ID NO: 1 - protein, 24 aa, Artificial Sequence
  Affinity tag
DSTIYASYYECGHGLSTGGYGYDS

SEQ ID NO: 2 - protein, 19 aa, Artificial Sequence
  Affinity tag
DTSTWYRGYCGTNPNYFSY

SEQ ID NO: 3 - protein, 17 aa, Artificial Sequence
  Affinity tag
GWSSLGSCGTNRNRYNY

SEQ ID NO: 4 - protein, 12 aa, Artificial Sequence
  Affinity tag
GYRNYGQCATRY

SEQ ID NO: 5 - protein, 12 aa, Artificial Sequence
  Affinity tag
GYRNYGQSATRY

SEQ ID NO: 6 - protein, 18 aa, Artificial Sequence
  Affinity tag
TRKYVPVRFALDQSSYDY